(19) United States
(12) Patent Application Publication
(10) Pub. No.: US 2009/0130748 A1
(43) Pub. Date: May 21, 2009

(54) OPTOREACTOR

(75) Inventor: Christian Uphoff, Chiemgau (DE)

Correspondence Address: BOYLE FREDRICKSON S.C., 840 North Plankinton Avenue, MILWAUKEE, WI, 53203, US

(73) Assignee: Georg Fritzmeier GmbH & Co. KG, Grosshelfendorf (DE)

(21) Appl. No.: 12/091,008

(22) PCT Filed: Oct. 19, 2006
(86) PCT No.: PCT/DE06/01846

§ 371 Date: Jul. 1, 2008

(30) Foreign Application Priority Data
Oct. 19, 2005 (DE) 10 2005 050 414.0

(51) Int. Cl. C12M 1/00

(52) U.S. Cl. 435/299.1

(57) ABSTRACT

The invention relates to a bioreactor for the treatment of industrial or domestic effluent, comprising a container and a packing, wherein a potential difference forms between the packing and the container, which is provided with a device which prevents corrosion of the container as a result of the potential reversal due to the growth of a biofilm.

### BACKGROUND OF THE INVENTION

1. Field of the Invention

The present invention relates to a bioreactor for the treatment of fluids loaded with organic or inorganic pollutants.

2. Description of the Related Art

Bioreactors of this type are especially employed in small domestic treatment works serving primarily for the treatment of domestic effluent. Said small domestic treatment works usually are already existing multi-chamber plants which have been equipped with an additional biological stage. The treated effluent is either allowed to trickle or supplied to the nearest open waters after passing the small domestic treatment works.

Also drainage waters from dumpsites or composting plants can be treated by these bioreactors.

Bioreactors for this use are known, for instance, from the patent applications DE 103 30 959.4 or the application FR 2439. The bioreactors described there consist of a photo-catalytically coated container and a packing arranged inside the container, wherein the packing includes a micro-biotic mixture of photosynthetically active microorganisms and luminous bacteria.

The pollutants are decomposed by means of this micro-biotic mixture, wherein the interaction of photosynthetically active microorganisms and luminous bacteria is exploited—an exact description of the action of the mixture is found in the publications DE 100 62 812 and DE 101 49 447. If moreover, as described in the patent application DE 102 53 334, photo-sensitizers are provided, singlet oxygen and other radicals are formed which accelerate the decomposition of the pollutants.

But also the design of container and packing itself influences the rate and quality of decomposition. If, for instance as disclosed in FR2439, the photo-catalytic layer is applied in bands alternately with a diamond coating on the outer surface of the container wall, due to the potential difference formed between the photo-catalytic layer and the sorption surface of the packing the diamond coating acts as diamond electrode in the area of which hydroxyl radicals are formed which permit to decompose even hardly soluble pollutants, such as rheumatism agents, for instance.

It is a drawback of said known bioreactors, however, that during operation of the bioreactor a biofilm forms on the container and the container starts corroding.

It is therefore the object of the present invention to provide a bioreactor which prevents such corrosion.

### SUMMARY OF THE INVENTION

This object is achieved by a bioreactor for the treatment of fluids loaded with organic or inorganic pollutants, comprising a container having at least one recess for the passage of the fluid to be treated and a packing arranged inside the container, wherein the container and the packing are formed such that a potential difference forms, characterized in that furthermore a device is provided which is designed to keep the orientation of the potential difference constant.

The basis of the present invention is the finding that the orientation of polarity of the container and the packing is gradually compensated and even reversed during operation by reason of the formation of a biofilm on the container wall, whereupon radicals increasingly attack also the material of the container which then results in the corrosion to be prevented.

In order to prevent said compensation or reversal of polarity several possibilities are provided.

On the one hand, in a first especially advantageous embodiment the container and the packing can be galvanically isolated. This very simple possibility prevents an exchange of charges from taking place, which may result in a reversal of polarity. Such galvanic isolation can be very easily achieved, for instance, by the fact that the container is not made of stainless steel but of plastic material.

In a further preferred embodiment of the invention a power source applied to the container is provided and thus the potential of the container is kept constant. This is advantageous especially because already existing bioreactors can easily be equipped with this additional power source so that an expensive exchange of the bioreactor and thus long-term shutoff of the domestic treatment plant is not necessary. This power source can be, for example, a solar cell, a power pack or a capacitor. It is especially preferred when the voltage of the potential difference itself ensures maintenance of the potential.

Furthermore, as shown in a third embodiment, also a barrier layer, for instance made of water glass and arranged beneath the photo-catalytically active layer, can prevent a potential reversal from taking place. This solution is advantageous especially for bioreactors to be newly employed.

Another advantageous possibility consists in the use of a sacrificial anode preventing corrosion. The great advantage thereof is that the sacrificial anode is simply added to the existing bioreactor so that a conversion or else a shutoff of a bioreactor in operation are not necessary.

These, and other aspects and objects of the present invention will be better appreciated and understood when considered in conjunction with the following description and the accompanying drawings. It should be understood, however, that the following description, while indicating preferred embodiments of the present invention, is given by way of illustration and not of limitation. Many changes and modifications may be made within the scope of the present invention without departing from the spirit thereof, and the invention includes all such modifications.

### BRIEF DESCRIPTION OF THE DRAWINGS

Hereinafter the invention will be illustrated in more detail by way of drawings and the pertinent descriptions, in which:

FIG. 1 shows a schematic representation of a multi-chamber pit including a retrofitted biological stage;

FIG. 2 is a schematic front view of an exemplary bioreactor from prior art consisting of a container and a packing;

FIG. 3 is a schematic representation of a first embodiment of the bioreactor according to the invention; and

FIG. 4 is a schematic representation of a second embodiment of the bioreactor according to the invention.

### DETAIL DESCRIPTION OF THE DRAWINGS

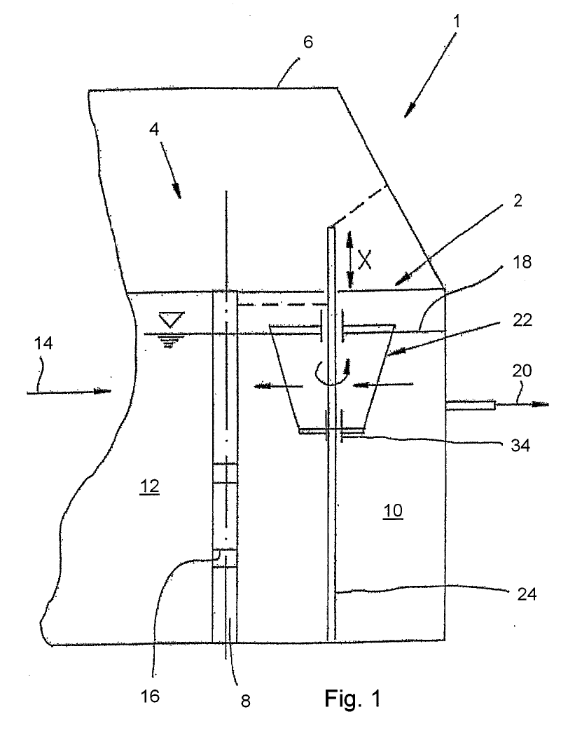

FIG. 1 shows a section across a small domestic treatment works 1 comprising a biological stage—i.e. a bioreactor 2 and a mechanical stage 4 formed by a three-chamber precipitation pit 4. On principle this is a chamber 6 which is subdivided by respective partitions 8 into three partial chambers of which merely a first chamber 10 and a further chamber 12 are shown in FIG. 1. The effluent to be purified flows toward the three-chamber precipitation pit through an inflow 14 and enters a first chamber—not shown—and can flow off through openings 16 in the walls 8 into the next partial chamber 12 and from there into the last partial chamber 10. In the individual chambers 10 and 12 settleable particles settle by sedimentation, while floating particles float on the liquid surface 18. The outlet 20 is selected such that the sediments and the floating particles remain in the chambers 10 and 12 and the purified effluent is discharged without these impurities. For biological preparation the bioreactor 2 constituting a biological stage is provided as retrofit kit in the chamber 10. The main component of this bioreactor is a container 22 in the form of a float in the shown embodiment, i.e. it has sufficient buoyancy that it floats in the effluent subject to biological treatment. For positioning the container 22 a vertical guide 24 which may be supported by the partition 8 and/or the side walls of the three-chamber precipitation pit 6 is provided in the chamber 10 (cf. broken lines in FIG. 1). The container 22 is movably arranged along this vertical guide 24 in the X direction in FIG. 1 so that depending on the liquid level 18 it is movable upward or downward as float inside the chamber 10.

A micro-biotic mixture forming a biofilm is introduced in the container 22. In the shown embodiment this micro-biotic mixture consists of a share of photosynthetically active microorganisms and a share of light-emitting microorganisms. The interaction between the photosynthetically active microorganisms and the luminous bacteria results in the fact that the photosynthetically active microorganisms are excited to perform photosynthesis by the emitted light of the luminous bacteria. The microorganisms run the photosynthesis with hydrogen sulphide and water as educt and release sulphur and/or oxygen. Furthermore, they can bind nitrogen and phosphate and decompose organic as well as inorganic material. As regards the concrete composition of this micro-biotic mixed culture, reference is made to the patent applications DE 100 62 812 A1 and DE 101 49 447 A1 of the applicant, to simplify matters. With reference to this application, only the essential steps of this photodynamic decomposition will be explained after description of the embodiments.

By interaction of the micro-biotic mixture as well as the catalytic surfaces of the container 22 a photodynamic decomposition of organic substances is brought about. This photodynamic decomposition of substances is described, for instance, in the application DE 102 53 334 of the applicant.

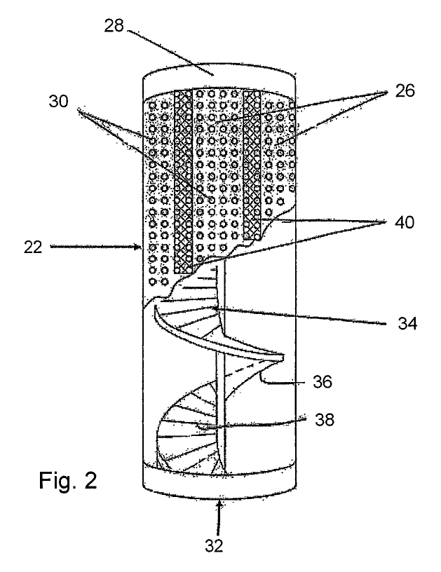

FIG. 2 shows another embodiment of the container 22. In this embodiment the container 22 is not funnel-shaped but cylindrical. The side walls of the container 22 are made of stainless steel and are partly provided with a photocatalytically active coating 26 in the shown embodiment. This coating can be formed at the inner circumferential wall of the container 22 and/or—as shown in FIG. 2—at the outer wall 28. In the shown embodiment the container 22 is made of V4A and is provided with a titanium oxide coating. Instead of this titanium dioxide, also indium tin oxide or the like can be used. The outer wall 28 of the container 22 is provided with a plurality of breakthroughs 30 so that the effluent subject to biological stabilization can enter the interior of the container 22. These breakthroughs 30 can be punched, for instance, wherein it is advantageous when the punching burrs project inwardly so that in this area slight growth of microorganisms can take place. The lower end face 32 of the container is closed so that the effluent flows into the container 22 substantially in radial direction. The upper end face can equally be closed. In case that this upper surface is located above the liquid level, closing can be renounced.

In the interior of the container 22 an exchangeable packing 34 is accommodated which has a spiral-shaped structure, as shown in the front view. In the shown embodiment this packing 34 consists of a carrier 36 which can be, for instance, a spiral-shaped stainless steel sheet. To this helical stainless steel carrier 36 a foam material, for instance PU foam coated or provided with active charcoal and nano composite material where appropriate, is applied on both sides. A pore system the walls of which are coated with active charcoal is formed by the PU foam so that a large material exchange area is made available.

Concretely, in the shown embodiment the carrier 36 consists of a VA grid member of two to three millimeters in thickness, the helical structure being formed by two grid surfaces between which half-hard, open-cell PU foam including an active charcoal coating is introduced. The grid bars 38 disposed on the downward directed side of the helix are provided with a photocatalytic surface, the mesh size at these downward directed large areas is approx. 10 to 12 mm. No coating is provided at the grid bars forming the upward directed large area of the helix. In this case the mesh size is about 25 to 30 mm.

The microorganisms mentioned in the beginning can be introduced into the center of the spiral-shaped packing 34 centrally via a dosing tube. However, it is also possible to introduce these microorganisms including the nano composite materials into the pore system already during manufacture of the packing 34. Tests in which the microorganisms and nano composite materials are dissolved in chitosane and this mixture to which nano composite materials have been added is then applied to the packing—for example by impregnation—so that continuous supply of microorganisms is dispensed with and merely at regular intervals an exchange of the packing 34 is required.

The PU foam is coated with a gel-like material of chitosane in the embodiment shown here on the downward directed side of the helix. The nano composite materials representing a piezoelectric ceramic system of PZT (lead zirconium titanate) short fibers having photocatalytic coatings are embedded in said chitosane. Furthermore, microorganisms typical of sewage treatment works and working on a biophysical basis are also embedded. On the upper side of the PU foam core only aerobic microorganisms are incorporated in the cationically active chitosane lactate.

The photodynamic decomposition of the organic parts is further assisted by the photocatalytic coating of the container 22. To this effect, the container 22 is coated both at its inner surface and at its outer surface with the photocatalytically active layer 26—namely titanium oxide, for instance. This layer is completely applied to the inner surface, i.e. the side facing the packing 34, whereas titanium dioxide is applied to the outer surface in the form of bands 26 between which areas provided with a diamond coating 40 are retained.

Such diamond coating 40 can be synthetically prepared by heating methane and hydrogen as well as an appropriate carrier of niobium, silicon or ceramic, for instance, in a vacuum chamber to temperatures of approx. up to 2000°. Then a reaction takes place in which a diamond lattice is formed on the carrier. This coating 40 is then applied to the outer wall 28 of the container 22 so that areas provided with a photocatalytically active layer 26 and with a diamond layer 40 are juxtaposed. These areas 26, 40 extend in longitudinal direction of the container 22. In the shown embodiment the width of the bands 26 corresponds approximately to the distance of four hole-shaped breakthroughs 30, while the width of the areas 40 is substantially smaller and corresponds approximately to the distance between two adjacent breakthroughs 30.

During interaction with the catalytic coating of the container 22 and the afore-described coating of the helical packing 34 a comparatively strong electromagnetic field is formed. The potential difference occurring is applied to the areas provided with the diamond coating 40 which then act as diamond electrodes. This voltage causes in the area of the diamond electrodes (areas 40) the formation of hydroxyl radicals which transform substances hardly or not decomposable so far into innocuous salts or carbon dioxide which is discharged in the form of gas overhead from the basket. That is to say, in the bioreactor according to the invention processes which result in an almost complete decomposition of the organic pollutants are run in parallel to each other by the interaction of the photocatalytically active layer and the biofilm on the packing as well as by the diamond electrodes. Details about the electromagnetic field formed are disclosed in the earlier application DE 103 30 959.4 so that further explanations in this respect are dispensable.

It is a problem, however, as already described in the foregoing, that the poling of the electromagnetic field can be influenced by the growth of the biofilm in such manner that the polarity of the container 22 and the packing 34 can be reversed. In this way the hydroxyl radicals which are nevertheless formed at the diamond coating do no longer migrate toward the pollutants—namely into the interior of the container toward the packing 34, but can also react with the stainless steel of the container which results in corrosion. This takes place even more when moreover the conductivity of the effluent is comparatively high (>1400 μS/cm), which is the case especially with a drainage water treatment from dumpsites.

In order to prevent such corrosion, hereinafter two especially advantageous embodiments of the bioreactors according to the invention will be discussed which are based on the bioreactor illustrated in FIG. 2 but do not admit polarity reversal.

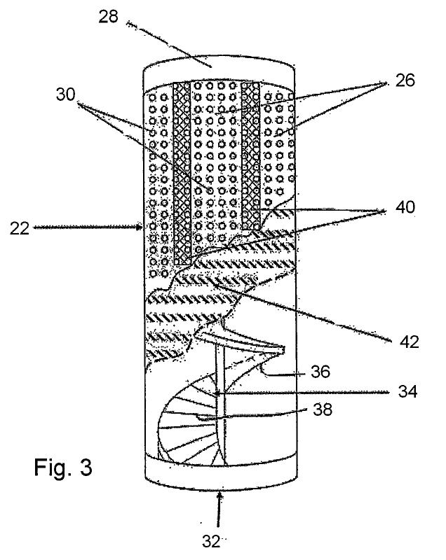

FIG. 3 shows a first embodiment of the bioreactor according to the invention. Between the inner wall of the container and the packing 34 a galvanic separation 42 is inserted. This separation prevents an exchange of charge between the packing 34 and the container 22 so that the polarity of the container 22 and the packing 34 is maintained. An especially simple galvanic separation would also consist already in manufacturing the container not of stainless steel but of plastic material.

It is also possible, however, to simply apply a barrier layer, for instance of water glass, which is located beneath the photocatalytic coating and the diamond coating as corrosion control.

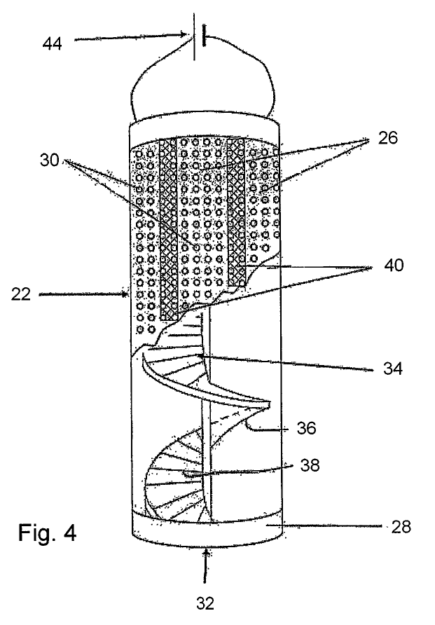

FIG. 4 shows a second embodiment of the bioreactor according to the invention. In this example the potential reversal is prevented by applying an external power source 44. The external power source 44 can be a power pack, a solar cell or a capacitor. It is especially preferred when the current produced by the bioreactor itself is utilized as power source.

It is especially advantageous when the power source 44 is operated at a maximum no-load voltage of 4.9 volt and a maximum current of 500 mA, because higher values may result in a destruction of the coating of the container. Under load a voltage of 1.5 to 2.2 volt and a current intensity of 500 mA are ideal so as to be able to ensure optimum operation of the bioreactor.

Instead of an external power source also a sacrificial anode can be employed. It consists of a material which is more susceptible to corrosion than the stainless steel of the container and thus has a higher attractive force for the highly reactive radicals.

The invention relates to a bioreactor for the treatment of industrial or domestic effluent, comprising a container and a packing, wherein a potential difference forms between the packing and the container, which is provided with a device which prevents corrosion of the container as a result of the potential reversal due to the growth of a biofilm.

Although the best mode contemplated by the inventors of carrying out the present invention is disclosed above, practice of the above invention is not limited thereto. It will be manifest that various additions, modifications and rearrangements of the features of the present invention may be made without deviating from the spirit and the scope of the underlying inventive concept.

1. A bioreactor for the treatment of fluids loaded with organic or inorganic pollutants, comprising a container having at least one recess for the passage of the fluid to be treated and a packing arranged inside the containers, wherein the container and the packing are formed such that a potential difference forms, characterized in that a device is provided which is designed to keep the orientation of the potential difference constant.

2. A bioreactor according to claim 1, wherein the device is a galvanic Separation between the container and the packing.

3. A bioreactor according to claim 3, wherein the container walls are coated with a photocatalytically active layer and the barrier layer is arranged beneath the photocatalytic layer.

4. A bioreactor according to claim 1, wherein the device is a power source connected to the containers.

5. A bioreactor according to claim 4, wherein the power source has a no-load voltage of approximately 4.9 volt.

6. A bioreactor according to claim 5, wherein under load the power source has a voltage of 1.5 to 2.2 volt.

7. A bioreactor according to claim 5, wherein the power source has a current intensity of 500 mA.

8. A bioreactor according to claim 5, wherein the power source is at least one of a group including a solar cell, a power pack and a capacitor.

9. A bioreactor according to claim 1, wherein the device is a sacrificial anode.

10. A bioreactor according to claim 1, wherein the device is a barrier layer especially made of water glass which is arranged on the container.

11. A bioreactor according to claim 1, wherein the container is made of plastic material.

12. A bioreactor according to claim 1, wherein the container is made of stainless steel.

13. A bioreactor according to claim 1, wherein the container walls are coated with a photocatalytically active layer, which is applied in sections at least to the outer circumferential surface, and a diamond coating is applied between the areas provided with the photocatalytically active layer.

14. A bioreactor according to claim 13, wherein the photocatalytically active layer and the diamond layer are applied in bands in the longitudinal direction of the container.

15. A bioreactor according to claim 1, wherein the packing comprises a micro-biotic mixture, preferably including a share of photosynthetically active microorganisms and a share of light-emitting microorganisms.

16. A bioreactor according to claim 12, wherein the stainless steel is V4A stainless steel.

17. A bioreactor according to claim 13, wherein the photocatalytically layer is one of a group including titanium dioxide and indium tin oxide.
